(19) United States
(12) Patent Application Publication
(10) Pub. No.: US 2004/0162487 A1
(43) Pub. Date: Aug. 19, 2004

(54) Medical examination and/or treatment system

(76) Inventors: Klaus Klingenbeck-Regn, Nuernberg (DE); Michael Maschke, Lonnerstadt (DE)

Correspondence Address: SCHIFF HARDIN, LLP, PATENT DEPARTMENT, 6600 SEARS TOWER, CHICAGO, IL, 60606-6473, US

(21) Appl. No.: 10/723,766

(22) Filed: Nov. 26, 2003

(30) Foreign Application Priority Data
Nov. 29, 2002 (DE) 102 559 57.0

(51) Int. Cl. A61B 5/05

(52) U.S. Cl. 600/427

(57) ABSTRACT

A medical examination and/or treatment system has an x-ray image acquisition system with a radiation source, a radiation receiver, as well as a control and processing device (with an image generation device) for controlling the source and receiver, a catheter system with an associated image acquisition system for optical coherence tomography, including a catheter with an optical fiber, via which light is guided to and radiated into the region of the catheter tip that is introduced into an examination region, and via which reflection light from the illuminated examination region is guided to a control and processing device with image generation device of the image acquisition system, as well as at least one monitor to present the x-ray images and coherence tomography images.

### BACKGROUND OF THE INVENTION

1. Field of the Invention

The present invention concerns a medical examination and/or treatment system.

2. Description of the Prior Art

One of the most common disorders, especially in industrial nations, is cardiac infarction (heart attack). This is caused by diseases of the arterial coronary vessels (atherosclerosis). Deposits (atherosclerotic plaque) on the vessel wall result in a reduction of the vessel diameter, culminating in a blockage (occlusion) of one or more coronary vessels. It has now been recognized that the danger of suffering a heart attack is not primarily dependent on the reduction of the vessel diameter. Rather, it also depends on whether the thin protective layer that covers the atherosclerotic deposit remains intact, If this layer ruptures, blood platelets preferentially attach at the breakage and close the vessel within a short time, and thus cause a heart attack.

For examination or treatment of such a condition, conventionally a catheter is used that is fed (advanced) into the region of the endangered coronary vessel under simultaneous x-ray supervision to acquire the position of the catheter and to monitor the examination/treatment. Such a catheter is manually inserted into the vascular system and advanced therein. Although the catheter is flexible, problems arise due to this manual guiding, above all in the region of vessel curves and the like. The movement and positioning of the catheter is consequently problematic. It can easily result in a perforation of the vessel wall if the tip of the catheter is pushed against it too forcefully, which can easily occur due to the limited possibilities of directional manipulation.

A further problem is that the simultaneous x-ray supervision provides only image information about the open (and thus unafflicted) volume of the vessel, but no information about the vessel wall (and thus the problematic plaque deposits) itself. Consequently, although known examination and/or treatment systems provide very important information for the physician, they do not provide all of the information required in the optimal case for a precise diagnosis.

### SUMMARY OF THE INVENTION

An object of the present invention is to provide an examination and/or treatment system that is improved with regard to addressing the above problems.

This object is achieved by an examination and/or treatment system in accordance with the invention having an x-ray image acquisition system having a radiation source, a radiation receiver, as well as a control and processing device (with an image generation device) that controls the radiation source and receiver, a catheter system with an associated image acquisition system for optical coherence tomography, having a catheter with an optical fiber, via which light is guided to and radiated into the region of the catheter tip introduced into an examination region, and via which reflection light from the illuminated examination region is guided to a control and processing device, with an image generation device, of the Image acquisition system for image generation as well as at least one monitor to show the x-ray images and coherence tomography images.

The Inventive system uses the known possibility to supervise the catheter movement as well as the vessel volume using an x-ray image acquisition system. Here, for example, a simple or a biplane C-arm device can be used. Furthermore, in the inventive system, a catheter system with associated image acquisition system is used that is fashioned to implement optical coherence tomography (OCT). In such a system (also called an OCT system), light is introduced (via an optical fiber arranged in the OCT catheter) that is decoupled in the region of the end of the introduced intravascular OCT catheter and illuminates the surroundings. This means the vessel to be examined (or the like) is illuminated from the inside. Via an optical fiber, light reflected from the vessel walls, etc. is conducted back and is processed in an image generation device of the image acquisition system in order to acquire an image of the inside of the vessel or the like on which the walls, possible deposits. etc. are visible. If many images are generated in succession, dependent on the type of camera acquisition a continuous image view of the inside of the examination region can ensue in a color representation. From this image, the operating physician has precise information about the appearance of the vessel wall that the physician can process together with the information from the parallel x-ray examination.

Overall, the inventive system offers a number of advantages, namely the acquisition of different images in the form of the x-ray images showing the open vessel volume and the OTC images displaying the detailed inner vessel wall and vessel structure. This means important image information, and items of image information to be combined with one another for the diagnosis, that are essential for the correct treatment, are made available to the physician. The x-ray images provide information about possible vessel narrowings, and thus the vessel diameter available for the blood flow, with very good image quality while the OCT images provide exact image information about the inner vessel wall and in particular about possible deposits or the atherosclerotic plaque and its surface. Naturally, the use of the inventive system also allows for examination, for example, of the heart chambers (atrium and ventricle).

In a further embodiment of the invention, the x-ray images and the OCT images can be shown simultaneously, in particular on a common monitor, allowing the physician to immediately assimilate both images (that show the same examination region) with one another, or compare and process both images with Image processing, insofar as this is necessary. A very advantageous superimposition of the x-ray images and the OCT images is also possible to show the examined vessel in the manner of an overall view, which allows the physician to visually combine and understand the information of both images.

Furthermore, it is convenient to employ a common image generation device to generate the x-ray images and the coherence tomography Images, such that appearance differences can be reduced.

In an embodiment of the invention at least one element that generates a magnetic field is provided at the catheter tip, and a device is provided to generate an external magnetic field serving to move the catheter inserted into the patient. In the inventive system, the catheter is not manually guided, but rather is guided by a magnetic field generated external to the patient. The magnetic field generated by the element at the catheter tip in the patient interacts with the external navigation or guide magnetic field that is modified (controlled) appropriately to move the catheter into the proper position with regard to the patient. In this manner it is possible to directionally control, approximately in real time, the catheter tip, that unavoidably must be guided around any vessel bends or the like, such that the catheter movement is substantially simpler and is possible with substantially more precision with regard to the positioning.

For a simple operation of the treatment system with its different components, it is convenient for the control device of the x-ray system and the control device of the image acquisition system and the control device of the device generating the external magnetic field to be integrated into a common control device, such that the operation of these subsystems can ensue from a single control center (console).

It is particularly convenient for the magnetic field of the catheter (which is generated via the magnetic field-generating element in the catheter tip) to be varied in a catheter inserted into a patient, thus can be varied with regard to its field strength and/or field direction. In this manner, the interaction of the catheter-generated magnetic field can be varied with the external magnetic field. Conveniently, an electromagnet with a core and a coil is used as the element generating the magnetic field, with the supply lines of the coil being carried in the catheter jacket and being conductively controlled from outside the patient via a catheter control device. In a simple manner, a field variation is possible via corresponding control of the coil current. By increasing the current, the field strength can be increased, by reversing the current direction the field direction also changes, etc. This control ensues in a simple manner via the catheter control device, that is fashioned as a separate device, for example a portable device, so that ft can be positioned relatively close to the patient, and long supply lines, at least to the coils, are not needed, It is of course also possible to integrate the catheter control device into the common control device.

It is convenient to provide two or more electromagnets in order to achieve, by superimposition of the individual fields in individually controllable electromagnets, a further degree of freedom with regard to the field variation. It is also possible to control the multiple electromagnets non-independently, but no necessarily in tandem.

In a further embodiment of the invention the (at least one) electromagnet is arranged such that the magnetic field generated thereby is substantially parallel to the longitudinal (lengthwise) axis of the catheter. In contrast to this, in an alternative version the (at least on) electromagnet Is arranged such that the magnetic field is substantially perpendicular to the longitudinal axis of the catheter, Depending on the field direction, a different interaction with the external field results (assuming no change in the basic field direction of the external field), meaning the thus generated force operating on the catheter tip is differently aligned.

In a particularly convenient embodiment at least two electromagnets are arranged such that the magnetic field generated by one of these electromagnets is substantially parallel to the catheter axis, and the magnetic field generated by the other electromagnet is substantially perpendicular to the catheter axis, such that both of the above possibilities can be used. Moreover, in the region of the catheter tip at least one permanent magnet element can be provided, such that this element, when Its magnetic field is sufficient for the motion, allows the operation of the electromagnet(s) to be at least temporarily foregone.

In an embodiment of the invention the catheter control device communicates with the control device to generate the external magnetic field, so the control of the electromagnet(s) ensues dependent on the control information if the magnetic field control device, This allows the catheter control device to react to changes of the current setting parameters of the magnetic field generation device, or these changes can be taken into account in the control of the coil current, such that the current supply to the coils can ensue depending on desired interaction.

For a good illumination of the vessel insides, it is convenient for the light to be radiated laterally from the fiber and/or forward from the tip. If the aforementioned magnets are present for automatic guiding, these can be disposed somewhat behind the tip, given a radiation directed forwards. If at all possible, omni-directional radiation is best in order to create the largest possible illumination region and in order to capture the most possible reflected light, such acquisition of that a large-area internal view acquisition is possible.

### DESCRIPTION OF THE DRAWINGS

FIG. 1 is a block diagram of the inventive examination and/or treatment device.

FIG. 2 is a section through a catheter and a block diagram of an associated catheter control device in accordance with the invention,

### DESCRIPTION OF THE PREFERRED EMBODIMENTS

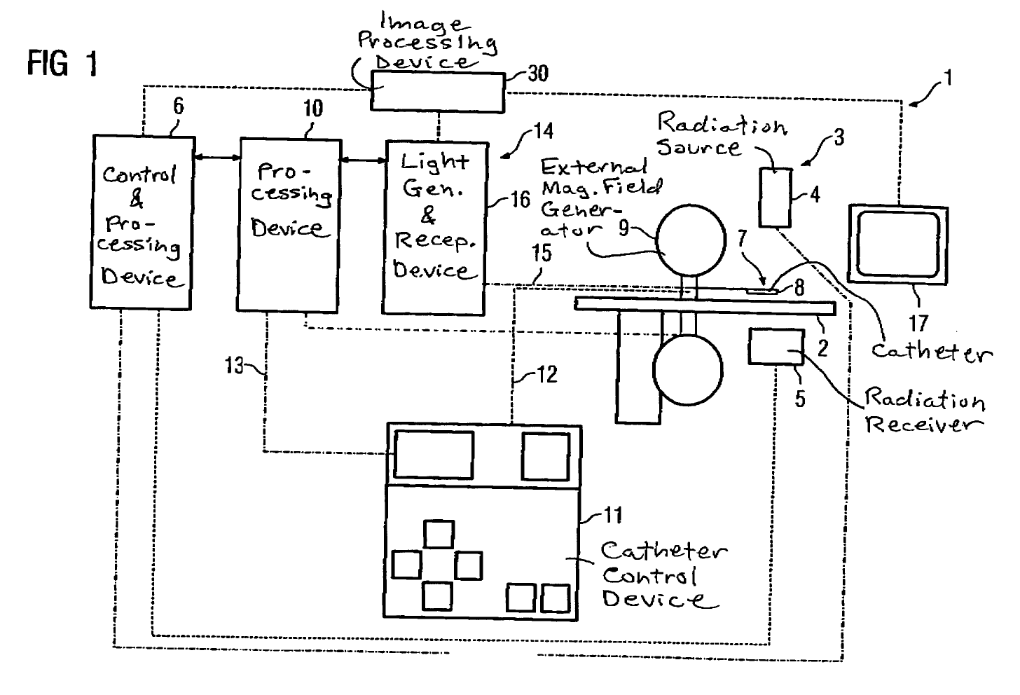

FIG. 1 shows an inventive examination and/or treatment device 1, by means of which a patient (not shown) located on a patient positioning table 2 can be examined/treated. The system has an x-ray image acquisition system 3 with a radiation source 4 to generate x-rays, a radiation receiver, for example a planar image detector 5 to acquire radiation images, as well as a control and processing device 6 that controls the operation of the radiation source 4, the radiation receiver 5 as well as the spatial movement and positioning, etc.

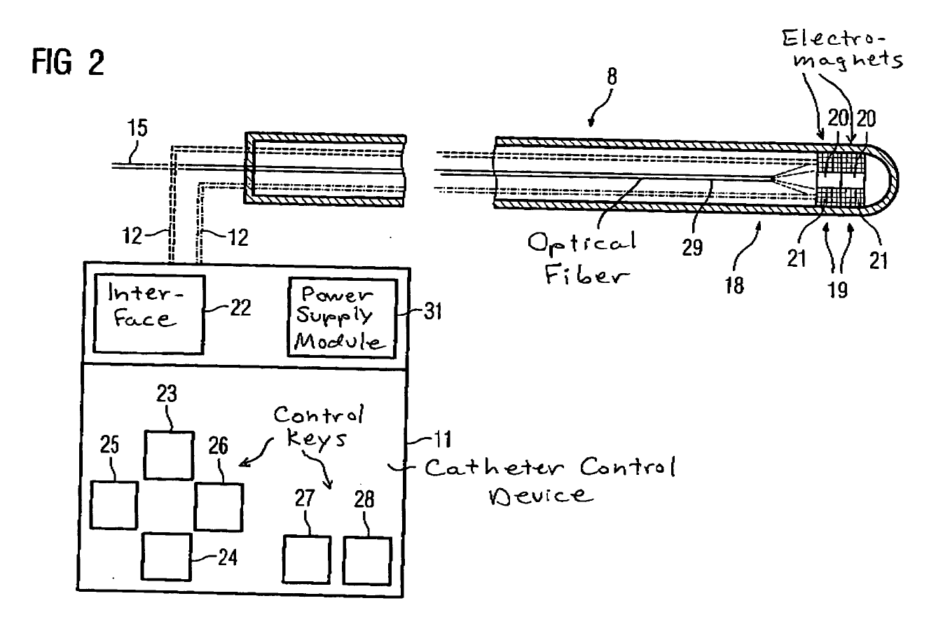

A catheter system 7 has an OCT catheter 8 to be inserted into a patient, with an element (which is shown in more detail in FIG. 2) arranged in its tip that generates a magnetic field. The catheter system 7 also includes, as well as a device 9 to generate a patient-external magnetic field that interacts with the magnetic field generated in the catheter tip in order to move the catheter through the vessel of the patient. Furthermore, the catheter system has a control and processing device 10, with which the operation of the device 9 generating the external magnetic field is controlled.

The catheter system 7 also has a catheter control device 11 that is connected via at least one supply connection 12 with at least one coil of an electromagnet (with which the catheter-side magnetic field is generated) arranged in the catheter tip, so the coil can be fed with current. Furthermore, the catheter control device 11 is connected for purposes of communication with the control and processing device 10 via a communication path 13, which can be hard-wired or wireless.

Furthermore, an image acquisition system 14 associated with the catheter system is provided, having a light generation and light reception device 16 (not shown in detail) that introduces light into a catheter-side optical fiber, and from this receives reflection light conducted back. The optical fiber connection 15 leads to the catheter 8.

The image signals acquired by the radiation receiver 5 and supplied to the control and processing device 6, as well as the reflection image signals or the reflection light received by the light generation and light reception device 16, are processed together in a digital image processing device 30. Furthermore, a monitor 17 is provided on which the x-ray images and OCT images generated by the image processing device 30 can be displayed, in particular together.

FIG. 2 shows the catheter 8 in detail, In the shown example, two electromagnets 19, each having a core 20 and a coil 21, are arranged in the catheter tip 18. Via the two supply lines 12, the coils 20 can be separately fed current, and consequently the electromagnets 19 can be separately operated.

The catheter control device 11 has an integrated power supply module 31 for feeding current. Furthermore, an interface 22 is provided, via which communication with the control and processing device 10 ensues for the external magnetic field generation device 9. This means that the current control information, based on which the external magnetic field is generated, and from which Its strength and direction and other relevant information can be acquired, is always available to the catheter control device 11. The current feed of the coils 20 can be controlled dependent on this information.

Due to the arrangement of both electromagnets 19, a generated magnetic field is always aligned in the direction of the lengthwise axis. Dependent on the direction of the coil current, the direction of the magnetic field can be reversed, by the poles being exchanged (reversed). Depending on how the external magnetic field is aligned with regard to the internal magnetic field, different interactions are possible. If the external magnetic field lies parallel to the internal magnetic field, depending on the field alignment a forward push or backward push can ensue by movement of the external magnetic field. The catheter follows the movement of the external magnetic field due to the magnetic interaction. This means that, when both fields are aligned the same, a quasi-longitudinal pushing motion ensues. If the external field is perpendicular to the internal field, the internal field attempts to rotate in the direction of the external field. This means that it is possible to initiate a turning or curving motion, i.e., a curve to the right or left or up or down, dependent on how the external and internal magnetic fields reside (i.e., their alignment to one another). The internal magnetic field always tries to rotate in the same direction as the external magnetic field.

In addition to the arrangement of the electromagnets shown in FIG. 2, it is also possible for the internal magnetic field to be substantially perpendicular to the catheter lengthwise axis. However, if the external magnetic field is likewise perpendicular to the catheter lengthwise axis, for example displaced by 90° to the internal magnetic field, a rotation motion around the catheter longitudinal axis can be initiated, since the internal magnetic field also tries to follow the external and to align identically therewith. This rotation motion ensues until the internal field is in identical alignment to the external. The known principle of an electromotor is used here to rotate the catheter by a short distance in a fixed external field. Naturally, a rotation to the left or right is also possible, depending on how the external field stands in relation to the internal field.

In order to arrange the internal field to produce the motion of the catheter desired by the physician, various operating elements (for example control keys 23, 24, 25, 26, 27, 28) are provided at the catheter control device 11. The key 23 stands for “forwards”, the key 24 for example for “backwards”. If, for example, the key 23 is pressed, the coil current is selected in a direction such that an internal magnetic field is generated that is aligned correspondingly identically to the external magnetic field such that, given a motion of the external magnetic field to the right, the catheter is, for example, likewise pushed to the right If the catheter should be pushed back, the polarity of the external magnetic field is reversed. The same is true for the internal magnetic field, which is automatically initiated by pressing the control key 24. The catheter control device 11 receives the information as to how to select the coil current via the interface 22, using the control information of the control and processing device 10.

The curving motion to the left or right ensues in a corresponding manner. This is initiated by pressing the operating element 25 or 26. For this, the internal field is correspondingly generated by selection of the current direction, using the knowledge of the direction of the external field perpendicular to the longitudinal axis of the catheter, such that the corresponding curve to the right or left results due to the interaction-conditional rectification of the internal field to the external field.

Should a rotation around the catheter lengthwise axis to the left or right be initiated, the corresponding operating elements 27, 28 are to be actuated. A further electromagnet (not shown more closely in a shown example) is controlled that is arranged perpendicular to the electromagnets 18, and that generates measurement data perpendicular to the catheter longitudinal axis. The selection of the coil current direction also ensues here dependent on the information concerning the external magnetic field or its alignment.

Furthermore, at the catheter the optical fiber 29, which was already mentioned above, is shown more closely. They end face thereof in the region of the catheter tip, where the radiated light from the catheter (to which, in this region, the catheter is transparent) is radiated into the surroundings. The necessary transparency can be obtained, for example, by using transparent synthetic materials to form the outer jacketing of the catheter, or by means of transparent elements in the outer catheter jacketing. The light can be radiated to the side, forward, or both to the side and forward, depending on the arrangement of the free end of the optical fiber 29. In the shown example, the radiation ensues to the side, meaning the catheter jacketing is transparent at least in the side area. The use of a number of optical fibers, possibly terminating at different locations, is also possible. The optical fibers 29 pass into the optical fiber connection 15, if necessary via a detachable plug connection.

To examine or treat a patient, the catheter is first inserted into the patient at a suitable insertion point; and it is subsequently guided by the external magnetic field given interaction with the internal magnetic field, and can thus be guided exactly to the examination area. This ensues under continuous x-ray supervision by means of the x-ray image acquisition system 3, with the x-ray images being displayed on the monitor 17. At the same time, during the entire movement path of the catheter, insertion or extraction, the OCT image acquisition can ensue via the OCT image acquisition system 14, meaning that continual knowledge about the concrete appearance of the inner vessel wall is also obtained. These OCT images are likewise shown on the common monitor 17.

Although modifications and changes may be suggested by those skilled in the art, it is the intention of the inventors to embody within the patent warranted hereon all changes and modifications as reasonably and properly come within the scope of their contribution to the art.

We claim as our invention:

1. A medical examination/treatment system comprising:
an x-ray image acquisition system comprising an x-ray source, an x-ray receiver disposed to receive x-rays from said x-ray source, and a control and processing device for controlling the x-ray source and the x-ray receiver, sad control and processing device generating an x-ray image from signals from said x-ray receiver;
a catheter system associated with an optical coherence tomography (OCT) image acquisition system, comprising a catheter with an optical fiber therein for guiding light from said OCT image acquisition system to a region of a tip of the catheter and for radiating the light from said region into an examination region at which reflection light is produced, and for guiding said reflection light from said region of the tip to a control and processing device of said OCT image acquisition system for generating an OCT image from said reflection light; and
at least one monitor at which said x-ray image and said OCT image are presented.

2. A medical examination/treatment system as claimed in claim 1 wherein said at least one monitor displays said x-ray image and said OCT image together.

3. A medical examination/treatment system as claimed in claim 2 wherein said at least one monitor is a single monitor at which said x-ray image and said OCT image are displayed superimposed.

4. A medical examination/treatment system as claimed in claim 1 wherein said control and processing device of said x-ray image acquisition system and said control and processing device of said OCT image acquisition system share a single image generator, and wherein said single image generator generates both said x-ray image and said OCT image.

5. A medical examination/treatment system as claimed in claim 1 wherein said catheter comprises a magnetic field-generating element disposed at said tip, and wherein said catheter system comprises a magnetic field generator that generates a magnetic field externally of said examination region that interacts with the magnetic field generated by said element to move said catheter relative to said examination region.

6. A medical examination/treatment system as claimed in claim 5 wherein said catheter system comprises a control unit for controlling at least one of said magnetic field generated by said element and said external magnetic field.

7. A medical examination/treatment system as claimed in claim 6 wherein said control and processing device of said x-ray image acquisition system, said control and processing device of said OCT image acquisition system, and said control unit of said catheter system are integrated into a single control device.

8. A medical examination/treatment system as claimed in claim 6 wherein said control device of said catheter system varies a strength of the magnetic field generated by said element.

9. A medical examination/treatment system as claimed in claim 8 wherein said magnetic field-generating element comprises an electromagnet having a core and a coil, and wherein said catheter comprises supply lines connecting said control device of said catheter system to said coil.

10. A medical examination/treatment system as claimed in claim 9 wherein said electromagnet is a first electromagnet, and wherein said catheter comprises a second magnetic field-generating element comprising a second electromagnet having a core and a coil, and wherein said catheter comprises second supply lines connecting said second electromagnet to said control device of said catheter system.

11. A medical examination/treatment system as claimed in claim 10 wherein said control device of said catheter system selectively controls said first and second electromagnets individually or in common.

12. A medical examination/treatment system as claimed in claim 5 wherein said catheter has a longitudinal axis, and wherein said magnetic field-generating element generates said magnetic field at said tip in a direction substantially parallel to said longitudinal axis.

13. A medical examination/treatment system as claimed in claim 5 wherein said catheter has a longitudinal axis, and wherein said magnetic field-generating element generates said magnetic field at said tip in a direction substantially perpendicular to said longitudinal axis.

14. A medical examination/treatment system as claimed in claim 5 wherein said catheter has a longitudinal axis, and wherein said magnetic field-generating element is a first magnetic field generating element that generates a first magnetic field substantially parallel to said longitudinal axis, and wherein said catheter comprises a second magnetic field-generating element, also disposed at said tip, that generates a second magnetic field substantially perpendicular to said longitudinal axis.

15. A medical examination/treatment system as claimed in claim 5 wherein said catheter comprises a permanent magnet element disposed in the region of said tip.

16. A medical examination/treatment system as claimed in claim 5 wherein said catheter system comprises a catheter control device connected to said magnetic field-generating element for varying the magnetic field generated by said magnetic field-generating element, and an external magnetic field control device for controlling said external magnetic field, and wherein said catheter system comprises a communication path between said catheter control device and said external magnetic field control device, and wherein said catheter control device controls said magnetic field generated by said magnetic field-generating element dependent on information supplied thereto by said external magnetic field control device regarding said external magnetic field.

17. A medical examination/treatment system as claimed in claim 1 wherein said optical fiber terminates in an end face adapted to radiate said light into said examination region from at least one location selected from the group consisting of sideways from said tip and forward from said tip.

18. A medical examination/treatment system as claimed in claim 1 wherein said control and processing device of said x-ray image acquisition system and said control and processing device of said OCT image acquisition system are integrated into a single control device.
